(19) United States
(12) Patent Application Publication
(10) Pub. No.: US 2009/0264987 A1
(43) Pub. Date: Oct. 22, 2009

(54) Stent Graft Delivery System and Method of Use

(75) Inventor: Adrian GALE, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/105,534

(22) Filed: Apr. 18, 2008

(51) Int. Cl. A61F 2/06

(52) U.S. Cl. 623/1.23; 623/1.11; 623/1.13

(57) ABSTRACT

A stent graft delivery system and method of use including a delivery system for a stent graft having a runner; a stent graft blank having at least one non-stented portion, the stent graft blank being positionable over the runner; and a stent graft cover having a stent graft cutter disposed in a distal end of the stent graft cover, the stent graft cover being slidably positionable over the stent graft blank. The stent graft cutter is heatable to cut the stent graft blank at the at least one non-stented portion to form the stent graft.

### TECHNICAL FIELD

The technical field of this disclosure is medical implantation devices, particularly, a stent graft delivery system and method of use.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include lumens such as those located within coronary, mesentery, peripheral, or cerebral vasculature; arteries; gastrointestinal tract; biliary tract; urethra; trachea; hepatic shunts; and fallopian tubes. Various types of endoluminal prostheses have also been developed with a particular structure to modify the mechanics of the targeted vessel wall.

A number of vascular devices have been developed for replacing, supplementing, or excluding portions of blood vessels. These vascular devices include endoluminal vascular prostheses and stent grafts. Aneurysm exclusion devices, such are used to exclude vascular aneurysms and provide a prosthetic lumen for the flow of blood. Vascular aneurysms (abnormal dilation of a blood vessel) are usually the result of disease or a genetic predisposition, which can weaken the arterial wall and allow it to expand. Aneurysms can occur in any blood vessel, but most occur in the aorta and peripheral arteries, with the majority of aneurysms occurring in the abdominal aorta or the aortic arch. An AAA (abdominal aortic aneurysm) typically begins below the renal arteries and extends into one or both of the iliac arteries. A TAA (thoracic aortic aneurysm) typically occurs in the ascending or descending aorta.

Aneurysms, especially abdominal aortic aneurysms, were historically treated with open surgery procedures where the diseased vessel segment is bypassed and repaired with an artificial vascular graft. While open surgery was and is an effective surgical technique in light of the high risk associated with a fatal abdominal aortic aneurysm rupture, the open surgical technique suffers from a number of disadvantages. It is complex, requires a long hospital stay, requires a long recovery time, and has a high mortality rate. Less invasive devices and techniques have been developed to avoid these disadvantages. Tubular endoluminal prostheses that provide a tubular graft for blood flow while excluding blood flow to the aneurysm site are introduced into the blood vessel using a catheter in a less or minimally invasive technique. The tubular endoluminal prosthesis is introduced in a small diameter compressed configuration and expanded at the aneurysm. Often referred to as stent grafts, these tubular endoluminal prostheses are used to secure tubular graft material held open in a sealing engagement with the vessel wall by one or more stents as a support structure.

Stent grafts for use in aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or holds it in place against a vessel wall. The stent graft is secured to a vessel wall above and below the aneurysm. An open crown without the graft material can be located above the aneurysm to provide a radial force to engage the vessel wall and seal the stent graft to the vessel wall.

One concern in the deployment of stent grafts is to assure that the stent graft is the proper length to cover the aneurysm, but not so long as to cover branching vessels, such as the renal arteries. Currently, the length of the stent graft is selected during pre-case planning for the anatomy of a particular patient from a limited number of available lengths. If the available length is unsuitable for the particular patient, the clinician must select the next best fit or commission expensive custom fabrication of a tailored stent graft. Additional problems can arise during surgery when the clinician finds that the selected stent graft is actually too short. The clinician must adjust the short stent graft so that it is functional or install additional stent grafts to fully line the aneurysm. Open surgical repair may even be required to remove the short stent graft.

It would be desirable to overcome the above disadvantages.

### SUMMARY OF THE INVENTION

One aspect according to the present invention provides a delivery system for a stent graft including a runner; a stent graft blank having at least one non-stented portion, the stent graft blank being positionable over the runner; and a stent graft cover having a stent graft cutter disposed in a distal end of the stent graft cover, the stent graft cover being slidably positionable over the stent graft blank. The stent graft cutter is heatable to cut the stent graft blank at the at least one non-stented portion to form the stent graft.

Another aspect according to the present invention provides a method of deploying a stent graft at a deployment site in a vessel, the method including advancing a stent graft blank to the deployment site, the stent graft blank being disposed over a runner and within a stent graft cover, a stent graft cutter being disposed in a distal end of the stent graft cover; retracting the stent graft cover until the stent graft cutter aligns with a desired non-stented portion of the stent graft blank; heating the stent graft cutter to cut the stent graft blank and to form a stent graft; and withdrawing the stent graft cover and remainder of the stent graft blank, the remainder of the stent graft blank being disposed within the stent graft cover.

Another aspect according to the present invention provides a delivery system for a stent graft including a runner having a runner nose and a runner body; a stent graft blank having a plurality of non-stented portions, the stent graft blank being positionable over the runner body; and a stent graft cover having a stent graft cutter disposed in a distal end of the stent graft cover, the stent graft cover being slidably positionable over the stent graft blank to retain the stent graft blank at a delivery diameter. The stent graft cutter is heatable with a radiofrequency beam to cut the stent graft blank at one of the plurality of non-stented portions to form the stent graft.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIGS. 1A-1C are schematic views of a stent graft cover, stent graft blank, and runner;

FIGS. 2A-2C are schematic side, detail side, and cross sectional views of a stent graft delivery system;

FIGS. 3A-3C are schematic views of stent graft deployment with a stent graft delivery system;

FIG. 4 is a side view of stent graft deployment in an abdominal aortic aneurysm with a stent graft delivery system;

FIG. 5 is another schematic view of stent graft deployment in an abdominal aortic aneurysm with a stent graft delivery system;

FIG. 6 is a schematic view of a stent graft delivery system;

FIG. 7 is a schematic view of another embodiment of a stent graft delivery system; and

FIG. 8 is a flowchart of a method of deploying a stent graft at a deployment site in a vessel.

### DETAILED DESCRIPTION

Embodiments according to the invention will now be described by reference to the figures wherein like numbers refer to like structures. The terms “distal” and “proximal” for the delivery system are used herein with reference to the treating clinician during the use of the stent graft delivery system: “distal” indicates a portion of the stent graft delivery system distant from, or a direction away from the clinician and “proximal” indicates a portion of the stent graft delivery system near to, or a direction towards the clinician. The terms “distal” and “proximal” for the stent graft are used herein with reference to the direction of blood flow from the patient's heart to and through the stent graft device: proximal” indicates a portion of the stent graft nearest the heart according to the blood flow path from the heart to the device, “distal” indicates a portion of the stent graft distant from heart according to blood flow path, or the end opposite the proximal end. In the example provided here, the proximal end of the stent graft during delivery corresponds with the distal end of the stent graft delivery system. As defined herein, the deployment site is the axial position in a vessel at which the proximal end of a stent graft is to be located when the stent graft is deployed.

Embodiments according to the invention disclose stent graft delivery devices and methods of use. While these devices and methods are described below in terms of being used to treat abdominal aortic aneurysms and thoracic aortic aneurysms, those skilled in the art will appreciate that the devices could be used to deliver other devices in other vessels as well. Stent graft delivery devices described include stent graft delivery systems for delivering a stent graft to a deployment site in a vessel, with the systems including a spindle fitting and stent capture fitting axially slidable relative to a nosecone shaft and releasably retaining the proximal (in these examples) end of the stent graft at a delivery diameter.

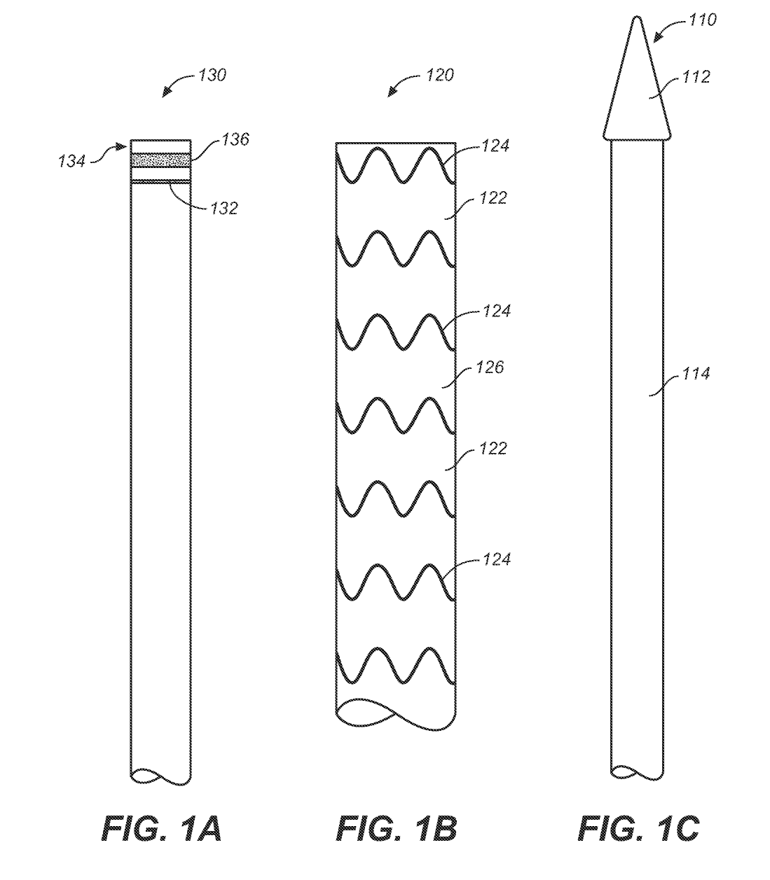

FIGS. 1A-1C are schematic views of a stent graft cover, stent graft blank, and runner, respectively, forming a stent graft delivery system. The delivery system includes a runner 110, a stent graft blank 120 positionable over the runner 110, and a stent graft cover 130 slidably positionable over the stent graft blank 120. The stent graft blank 120 has at least one non-stented portion 122. A stent graft cutter 132 is disposed within the stent graft cover 130. The stent graft cutter 132 is heatable to cut the stent graft blank 120 at the non-stented portion 122 to form the stent graft. The stent graft cutter 132 is shown on the stent graft cover 130 for clarity of illustration, but is disposed within the stent graft cover 130 at distal end 134 (the wiring is not shown as the routing of wiring through and to the end of catherter structures is well known in the art).

Referring to FIG. 1C, the runner 110 supports the stent graft blank so that the stent graft blank can be delivered to a deployment site in a vessel. In one embodiment, the runner 110 includes a runner nose 112 and a runner body 114. The runner nose 112 can be generally tapering from the distal to the proximal end to facilitate passage through a vessel. The diameter of the proximal end of the runner nose 112 can be the same as the outer diameter of the stent graft cover to provide a smooth surface for the assembled stent graft delivery system. The runner body 114 is long enough to reach from the deployment site in the vessel to the clinician. In one embodiment, the runner 110 can include a guide wire lumen. The runner 110 can be made of a single material, or the runner nose 112 and the runner body 114 can be made of different materials. The runner 110 can be made of flexible biocompatible materials. For example, the runner 110 can be made of polyurethane, polyethylene, PEBAX, nylon, or the like. The runner nose 112 can include a radiopaque additive to provide the clinician with a visible tip when using fluoroscopy guidance to deliver the stent graft within the patient.

Referring to FIG. 1B, the stent graft blank 120, illustrated in the expanded state, includes stents 124 and graft material 126 supported by the stents 124. The non-stented portions 122 are portions of the stent graft blank 120 without stents 124. Any of the non-stented portions 122 can be cut with the stent graft cutter. In this example, the stent graft blank 120 is a single tube with regularly spaced stents 124. The single tube can be the main stent graft or can be an iliac limb, an aorta extender cuff, or an iliac extender cuff. In another embodiment, the stent graft blank 120 is a bifurcated tube. In another embodiment, the stent graft blank 120 includes a bare spring extending distally beyond the graft material 126 to provide a radial force which engages the vessel wall and seals the stent graft at the vessel wall. In another embodiment, the stents 124 of the stent graft blank 120 are irregularly spaced. The stent graft blank 120 is delivered to the deployment site at a delivery diameter and expanded at the deployment site to a deployed diameter.

The stent graft formed from the stent graft blank 120 can be described as any suitable device for mechanically keeping a tubular graft open and in sealing contact with healthy surrounding tissue after being implanted at the deployment site, such as a deployment site in the abdominal aorta, thoracic aorta, or other vessel. Such mechanical endoprosthetic devices are typically inserted into the target vessel, positioned across the lesion, and then expanded to bypass the weakened wall of the vessel, thereby preventing rupture of the aneurysm. The stent graft is in contact with the healthy tissue after implantation of the stent graft. The stent graft generally extends across the aneurysm in a vessel to divert flow through the stent graft and relieve the pressure normally applied to the weak aneurysmal wall.

The size and configuration of the stents 124 depend upon the size and configuration of the vessel to be treated. Some of the individual stents 124 can be connected to each other by articulated or rigid joints as long as non-stented portions are provided. The minimum length of the stent graft blank 120 is the length of the aneurysm across which the stent graft will be implanted plus an additional remainder to assure that the stent graft blank 120 is longer than the aneurysm. A remainder of the stent graft blank 120 is discarded after the stent graft is formed from the stent graft blank 120.

The stents 124 and the graft material 126 can be any stents and the graft material typically used for stent grafts. The stents 124 can be self-expanding. The stents 124 can be made of can be made of spring steel, stainless steel, titanium, nickel titanium alloys (Nitinol), a polymer or copolymer, a combination of these materials, or other suitable materials. The graft material 126 can be any woven or interlocked graft material suitable for stent grafts, such as woven polymer materials, e.g., Dacron polyester, or polytetrafluoroethylene (PTFE), or interlocked graft materials including knit, stretch, and velour materials. In some embodiments, the graft material 126 includes components made of collagen, albumin, an absorbable polymer, or biocompatible fiber. Alternatively, the graft material 126 is constructed from one or more suitable plastic or non-biodegradable materials.

Referring to FIG. 1A, the stent graft cover 130 is an elongate tube which retains and/or compresses the stent graft blank 120 on the runner 110 when the stent graft blank 120 is being delivered to the deployment site in the patient. The stent graft cover 130 is then retracted to allow the distal portion of the stent graft blank 120 to expand at the deployment site. The stent graft cutter 132 disposed within the stent graft cover 130 is used to cut the stent graft blank 120 to the desired length to form the stent graft. The stent graft cover 130 can also include a radiopaque marker 136 at the distal end 134 to locate the stent graft cover 130 in the vasculature and locate the stent graft cutter 132 relative to the stent graft blank 120. The stent graft cover 130 can be made of flexible biocompatible materials. For example, the stent graft cover 130 can be made of polyurethane, polyethylene, PEBAX, nylon, or the like.

The stent graft cutter 132 is located on the inside circumference of the stent graft cover 130. The stent graft cutter 132 can be molded into the stent graft cover 130 or attached to the stent graft cover 130 with an adhesive. The adhesive can be any biocompatible, thin, high bonding adhesive. An insulator can be placed between the stent graft cutter 132 and the stent graft cover 130 to protect the stent graft cover 130 from heat from the stent graft cutter 132 during cutting. In one embodiment, a polyxylene polymer such as Parylene can be used as the insulator. The polymer can also be used around the stent graft cutter 132 to control and direct the heat from the stent graft cutter 132. For example, the polymer can cover most of the stent graft cutter 132, such as 80 percent of the surface area, leaving a small ring of the stent graft cutter 132 exposed to the stent graft blank, such as 20 percent of the surface area. The small ring which is exposed provides the heat to cut the stent graft blank.

The stent graft cutter 132 can be formed of any material which can generate sufficient heat to cut the stent graft blank. The stent graft cutter 132 can be a single piece or multiple turns of wire. In one embodiment, the stent graft cutter 132 is heated with a radiofrequency (RF) source, such as an RF source delivering 180 to 300 Watts, applying a radiofrequency beam to the stent graft cutter 132 from outside the patient. The stent graft cutter 132 can be made of any material that can be heated by RF, such as metal or ceramic composites. For example, the stent graft cutter 132 can be made of nitinol, stainless steel, or the like. In another embodiment, the stent graft cutter 132 is heated with a current source wired to the stent graft cutter 132 passing an electric current through the stent graft cutter 132. The wires to the stent graft cutter 132 from the current source follow the stent graft cover 130 to the outside of the patient. The stent graft cutter 132 can be made of any material that can be heated with an electrical current, such as metal or ceramic composites. For example, the stent graft cutter 132 can be made of nitinol, stainless steel, nichrome, or the like, and the current source can be an electrocautery power supply. The combination of stent graft cutter 132 and graft material can selected so that the stent graft cutter 132 seals the edge of the graft material when making the cut. Cutting is initiated by the energization of the stent graft cutter so that it is heated to melt the surrounding graft material structure.

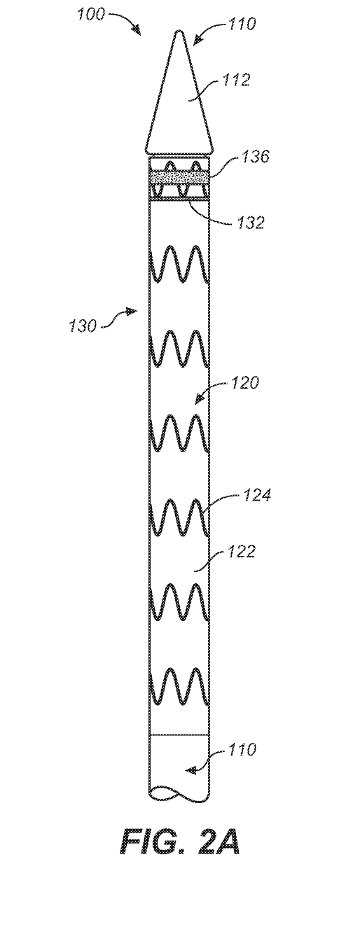

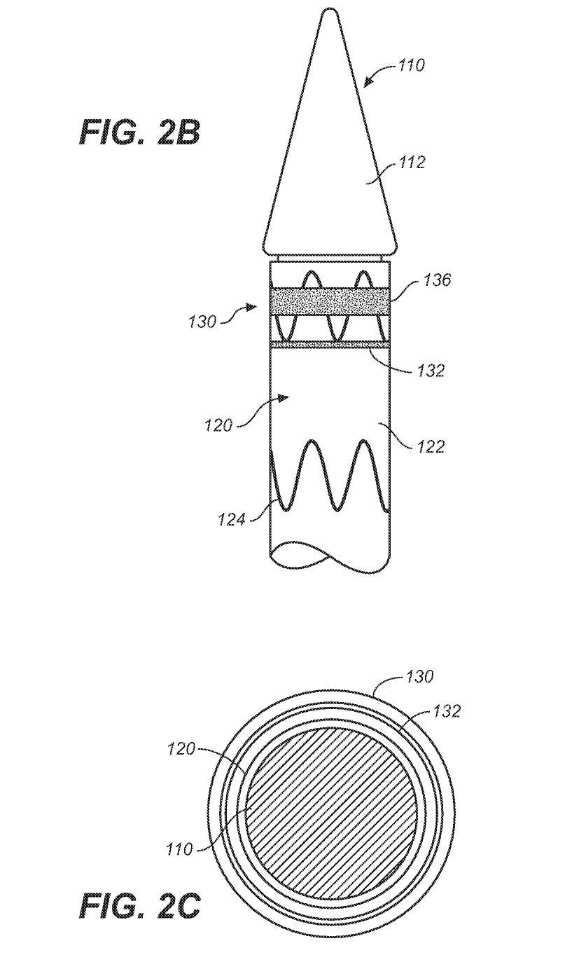

FIGS. 2A-2C are a side view, detail side view, and cross section view of a stent graft delivery system. For clarity of illustration, the runner 110, stent graft blank 120, and stent graft cutter 132 are shown as visible within the stent graft cover 130. The cross section view of FIG. 2C is taken across the stent graft cutter 132. The assembled delivery system 100 includes the stent graft blank 120 retained and/or compressed over the runner 110 by the stent graft cover 130. The proximal end of the delivery system 100 includes a handle (not shown) for manipulation by the clinician during stent graft delivery which retracts the stent graft cover 130 relative to the runner 110 without withdrawing the stent graft blank 120 relative to the runner 110. An exemplary handle is the Xcelerant delivery system from Medtronic, Inc., which provides both a slow rotational retraction and a quick release retraction.

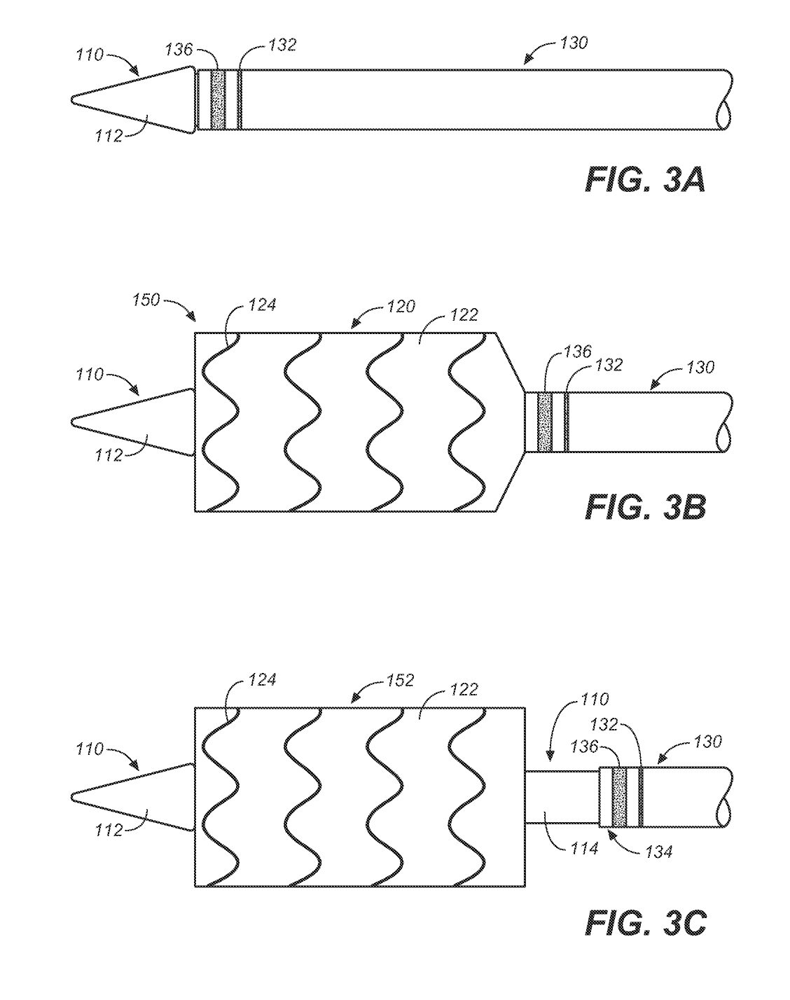

FIGS. 3A-3C are side views of stent graft deployment with a stent graft delivery system. Referring to FIG. 3A. The delivery system 100 is advanced to the deployment site, such as across an aneurysm, with the stent graft blank 120 at a delivery diameter, disposed over the runner 110 and within the stent graft cover 130. Referring to FIG. 3B, when the distal end 150 of the stent graft blank 120 is aligned at the desired portion of the deployment site, the stent graft cover 130 is retracted until the stent graft cutter 132 aligns with a desired non-stented portion 122. The desired non-stented portion 122 can be selected by the clinician to be long enough to completely cover the aneurysm while being short enough to avoid interfering with other anatomy, such as the iliac bifurcation. The portion of the stent graft blank 120 which is uncovered as the stent graft cover 130 is retracted expands to the deployed diameter. The stent graft cutter 132 is heated to cut the stent graft blank 120 at the desired non-stented portion 122 and to form the stent graft 152. In one embodiment, the stent graft cutter 132 is heated with an RF source outside the patient. In another embodiment, the stent graft cutter 132 is heated with an electric current from a current source wired to the stent graft cutter 132. Referring to FIG. 3C, the stent graft 152 is deployed in the aneurysm. The stent graft cover 130 and remainder of the stent graft blank 120 disposed within the stent graft cover can be withdrawn. In one embodiment, the runner nose 112 of the runner 110 can be withdrawn through the lumen of the stent graft 152 until the runner nose 112 is against the distal end 134 of the stent graft cover 130, then the runner 110 and the stent graft cover 130 can be withdrawn together as a single unit.

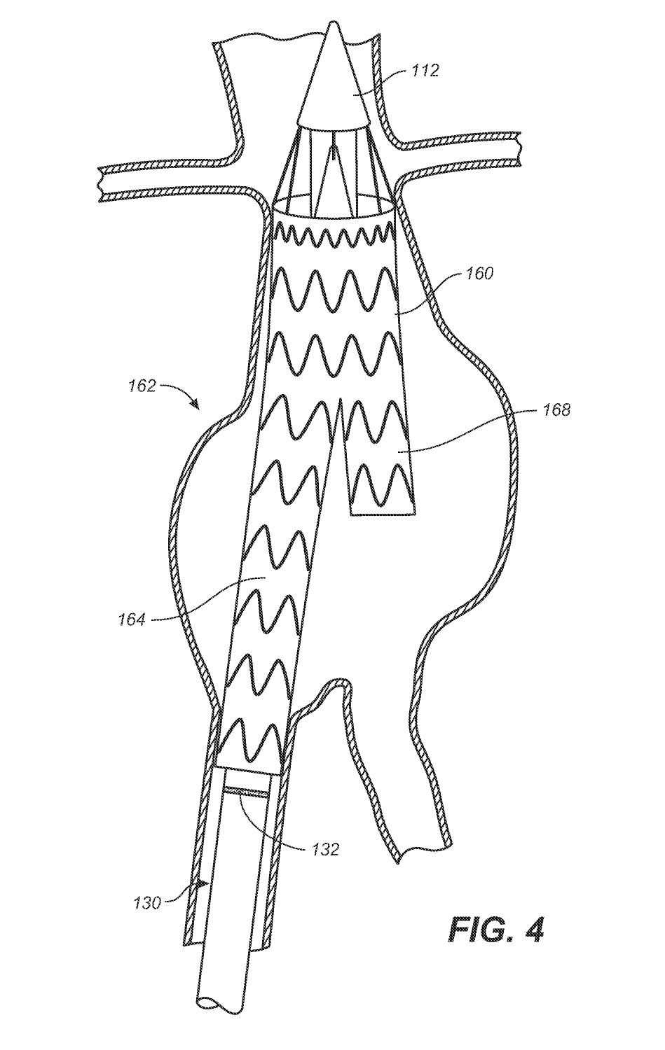

FIG. 4 is a side view of stent graft deployment in an abdominal aortic aneurysm with a stent graft delivery system. In this example, the stent graft 160 is a bifurcated stent graft deployed across an aneurysm 162 as the deployment site. The ipsilateral limb 164 has been cut to length with the stent graft cutter 132 on the stent graft cover 130. Because the length is determined in situ, the length is tailored to the particular aneurysm in the particular patient. The iliac limb installed in the contralateral limb 168 can also be formed from a stent graft blank cut to length with a stent graft cutter on a stent graft cover.

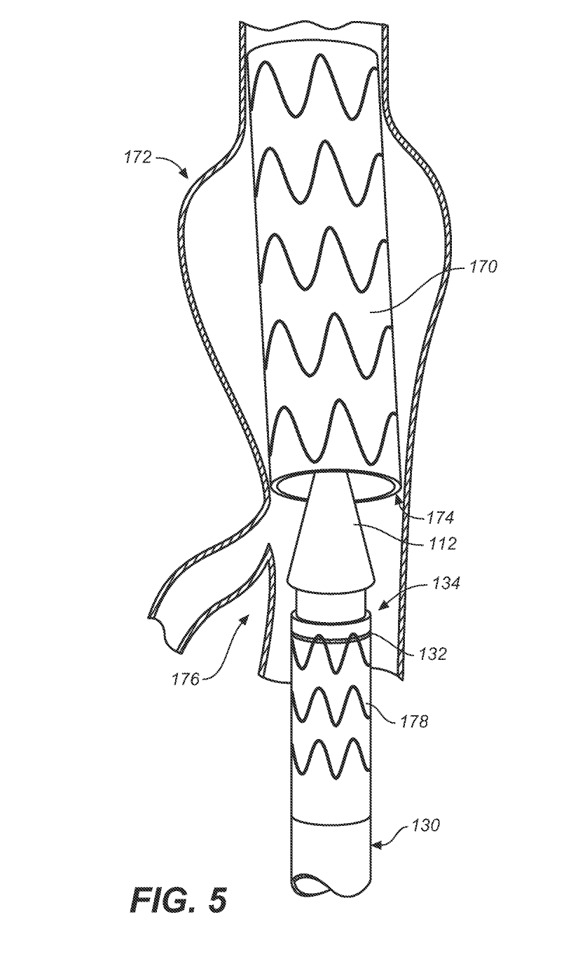

FIG. 5 is a another side view of stent graft deployment in an abdominal aortic aneurysm with a stent graft delivery system. In this example, the stent graft 170 across the aneurysm 172 is cut to length so that the proximal end 174 of the stent graft 170 falls short of the iliac bifurcation 176, keeping both iliac arteries open. The remainder 178 of the stent graft blank, which is shown as visible within the stent graft cover 130 for clarity of illustration, remains within the stent graft cover 130. The runner nose 112 is withdrawn to the distal end 134 of the stent graft cover 130 so the runner 110 and the stent graft cover 130 can be withdrawn together as a single unit.

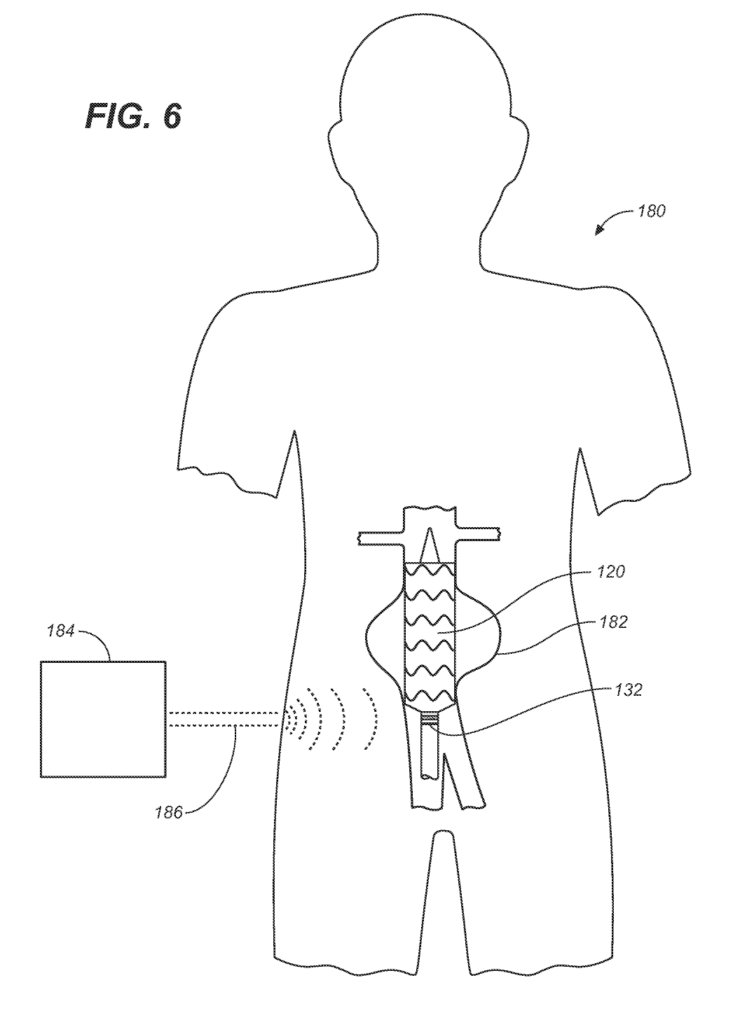

FIG. 6 is a schematic view of a stent graft delivery system. In this embodiment, the stent graft cutter 132 in the patient 180 is heated by a radiofrequency (RF) beam 186 from an RF source 184. In one example, the RF source 184 delivers 180 to 300 Watts power.

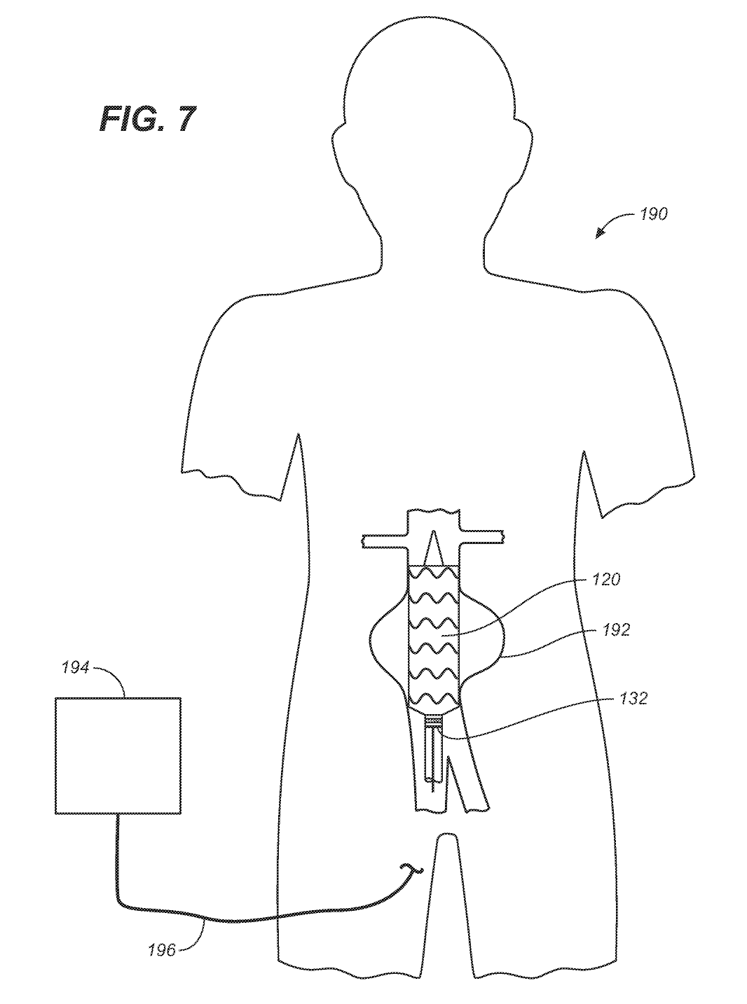

FIG. 7 is a schematic view of another embodiment of a stent graft delivery system. In this embodiment, the stent graft cutter 132 in the patient 190 is heated by an electric current from a current source 194 attached to the stent graft cutter 132 with a wire 196. Within the patient 190, the wire 196 follows the stent graft cover to the stent graft cutter 132. In one embodiment, the current source is an electrocautery power supply.

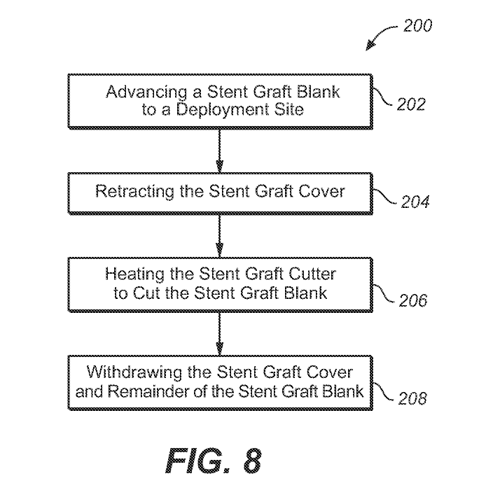

FIG. 8 is a flowchart of a method of deploying a stent graft at a deployment site in a vessel. The method (200) includes the steps of advancing a stent graft blank to the deployment site (202); retracting the stent graft cover (204) until the stent graft cutter aligns with a desired non-stented portion; heating the stent graft cutter to cut the stent graft blank (206) and to form a stent graft; and withdrawing the stent graft cover and remainder of the stent graft blank (208). A stent graft cutter is disposed in a distal end of the stent graft cover. The stent graft blank is disposed over a runner and within a stent graft cover when advancing a stent graft blank to the deployment site (202). The remainder of the stent graft blank is disposed within the stent graft cover when withdrawing the stent graft cover and remainder of the stent graft blank (208). In one embodiment, withdrawing the stent graft cover and remainder of the stent graft blank (208) also includes withdrawing the runner simultaneously with the stent graft cover and the remainder of the stent graft blank.

Heating the stent graft cutter to cut the stent graft blank (206) can include applying a radio frequency beam to the stent graft cutter. In another embodiment, heating the stent graft cutter to cut the stent graft blank (206) can include passing an electric current through the stent graft cutter.

While specific embodiments according to the invention are disclosed herein, various changes and modifications can be made without departing from its spirit and scope.

1. A delivery system for a stent graft comprising:
a runner;
a stent graft blank having at least one non-stented portion, the stent graft blank being positionable over the runner; and
a stent graft cover having a stent graft cutter disposed in a distal end of the stent graft cover, the stent graft cover being slidably positionable over the stent graft blank;
wherein the stent graft cutter is heatable to cut the stent graft blank at the at least one non-stented portion to form the stent graft.

2. The delivery system of claim 1 wherein the runner comprises a runner nose and a runner body proximal of the runner nose.

3. The delivery system of claim 1 wherein the stent graft blank is a single tube.

4. The delivery system of claim 1 wherein the stent graft blank is a bifurcated tube.

5. The delivery system of claim 1 wherein the stent graft cover is sized to compress the stent graft blank.

6. The delivery system of claim 1 wherein the stent graft cutter is made of a material selected from the group consisting of nitinol and stainless steel.

7. The delivery system of claim 1 further comprising a radiofrequency source operable to provide a radiofrequency beam to the stent graft cutter.

8. The delivery system of claim 1 further comprising a current source wired to the stent graft cutter to provide an electric current to the current source.

9. The delivery system of claim 1 further comprising a radiopaque marker at a distal end of the stent graft cover.

10. A method of deploying a stent graft at a deployment site in a vessel, the method comprising:
advancing a stent graft blank to the deployment site, the stent graft blank being disposed over a runner and within a stent graft cover, a stent graft cutter being disposed in a distal end of the stent graft cover;
retracting the stent graft cover until the stent graft cutter aligns with a desired non-stented portion of the stent graft blank;
heating the stent graft cutter to cut the stent graft blank and to form a stent graft; and
withdrawing the stent graft cover and remainder of the stent graft blank, the remainder of the stent graft blank being disposed within the stent graft cover.

11. The method of claim 10 wherein the heating the stent graft cutter comprises applying a radio frequency beam to the stent graft cutter.

12. The method of claim 10 wherein the heating the stent graft cutter comprises passing an electric current through the stent graft cutter.

13. The method of claim 10 further comprising withdrawing the runner simultaneously with the stent graft cover and the remainder of the stent graft blank.

14. A delivery system for a stent graft comprising:
a runner having a runner nose and a runner body;
a stent graft blank having a plurality of non-stented portions, the stent graft blank being positionable over the runner body; and
a stent graft cover having a stent graft cutter disposed in a distal end of the stent graft cover, the stent graft cover being slidably positionable over the stent graft blank to retain the stent graft blank at a delivery diameter;
wherein the stent graft cutter is heatable with a radiofrequency beam to cut the stent graft blank at one of the plurality of non-stented portions to form the stent graft.
